(19) United States
(12) Patent Application Publication
(10) Pub. No.: US 2025/0389972 A1
(43) Pub. Date: Dec. 25, 2025

(54) EYEGLASSES AND NEUROIMAGING DEVICE

(71) Applicant: Father Flanagan's Boys' Home Doing Business as Boys Town National Research Hospital, Omaha, NE (US)

(72) Inventors: Alexa Marie Wildy, Omaha, NE (US); Cooper Lee Livermore, Omaha, NE (US)

(21) Appl. No.: 19/246,007

(22) Filed: Jun. 23, 2025

Related U.S. Application Data
(60) Provisional application No. 63/663,497, filed on Jun. 24, 2024.

(51) Int. Cl. G02C 5/00; A61B 5/246; G02C 3/00

(52) U.S. Cl. CPC G02C 5/001; A61B 5/246; G02C 3/003; A61B 2560/06

(57) ABSTRACT

Neuroimaging and/or brain activity sensing machines, such as MEG and OPM machines use a helmet-like or head covering device that includes sensors for acquiring information from a participant's brain activity. The machine is operated while the participant undergoes tests, which can include a need for sight. People with glasses, therefore, need access to their glasses, but the glasses need to be non-ferromagnetic. In addition, due to the shape of the helmet-like objects and the need to have the sensors as close to the participant's head as possible, having glasses that include a contour that closely matches the shape of the helmet will improve the information obtained by the machine.

### CROSS REFERENCE TO RELATED APPLICATIONS

This application claims priority under 35 U.S.C. § 119 (e) to provisional patent application U.S. Ser. No. 63/663,497, filed Jun. 24, 2024. The provisional patent application is hereby incorporated by reference in its entirety herein, including without limitation: the specification, claims, and abstract, as well as any figures, tables, appendices, or drawings thereof.

### TECHNICAL FIELD

The present disclosure relates generally to the field of neuroscience. More particularly, but not exclusively, the disclosure includes peripherals and/or apparatus that can be used with neuroimaging machines to aid in acquiring the best data from use of the machine.

### BACKGROUND

The background description provided herein gives context for the present disclosure. Work of the presently named inventors, as well as aspects of the description that may not otherwise qualify as prior art at the time of filing, are neither expressly nor impliedly admitted as prior art.

OPM-MEG (Optically Pumped Magnetometers-Magnetoencephalography (MEG) is a new non-invasive technology for imaging brain function in real-time with several advantages over traditional MEG.

Magnetoencephalography (usually abbreviated as MEG) is a non-invasive technology for imaging brain function in real time. The approach is based on the measurement of magnetic fields generated (mainly) by synchronous dendritic current flow through neuronal assemblies. Therefore, it is a direct measurement of brain activity. Mathematical modelling of these fields permits the development of three-dimensional images (termed source reconstruction) depicting the moment-to-moment variations in electrical activity as the brain responds to different experimental circumstances or cognitive demands.

This technique is the only neuronal imaging device capable of recording brain activity with clinical accuracy for brain surgery or neurological disease diagnosis with millisecond-scale temporal resolution and spatial resolution of 2-5 mm. This brings an improved anatomical localization of pathological or functional brain regions over other related techniques such as ElectroEncephaloGraphy (EEG) whose spatial resolution is limited by skull-related distortions in electrical potential.

Additionally, MEG is a non-invasive imaging technique and is completely safe. It uses neither ionizing radiation nor injection. Consequently, it is particularly well suited to babies and the pediatric population.

The MEG machines include a helmet-like head portion in which a portion of a patient is positioned within the helmet-like head portion for placement at or near sensors. For both MEGs and OPMs, glasses are sometimes worn by patients/participants. For MEG and OPM, any glasses that are used with the machine must have no ferromagnetic metal. This means that regular glasses with hinges and screws cannot be used. Additionally, the normal shape of most glasses are too large and bulky to be able to fit comfortably while in the MEG/OPM. Most non-ferromagnetic glasses are made to be able to work with standard MRIs which do not have a shape restriction. Most MEGs have a smaller window that glasses must fit within which requires the glasses to have a sharp curve down on the top of the frames. For adults with smaller heads, their eyes are level with the MEG's rim, so the glasses need to sit low on the face. If the glasses obstruct the ability for the person's head to reach the top of the helmet of the scanner, the scanner is unable to get the best data. The last issue with regular glasses is the legs of the glasses. Often people being scanned in the MEG have head position indicator coils (HPI coils) taped behind their ears so glasses that have legs that bend around the ear may touch or move the HPI coils degrading the MEG signal.

Similarly, with OPMs, a helmet or other head covering apparatus is used to house the sensors measuring brain activity. The helmet or other head covering used with the OPM must fit far enough down on a patient's head that it can create issues with the use of glasses while undergoing testing with the machine.

There are many non-ferromagnetic glasses but a lot of them do not fit in the MEG. There are some that claim to fit in the MEG, but across the board these alternatives require the participant to have extremely specific head sizes, either small or tall. Small heads can fit with most frame shapes without issue due to the ample space to fit their head and the entirety of the frames in the helmet/head covering of the scanner. Participants with larger foreheads can often wear various frames because their heads reach the top of the scanner before their eyes are near the scanners rim. The frames on the market right now neglect those with medium to large head sizes and those with shorter to medium forehead length.

Thus, there exists a need in the art for an apparatus which allows a patient/participant to be able to wear glasses while undergoing testing with a neuroimaging machine, such as an MEG and/or OPM.

### SUMMARY

The following objects, features, advantages, aspects, and/or embodiments are not exhaustive and do not limit the overall disclosure. No single embodiment need provide each and every object, feature, or advantage. Any of the objects, features, advantages, aspects, and/or embodiments disclosed herein can be integrated with one another, either in full or in part.

It is a primary object, feature, and/or advantage of the present disclosure to improve on or overcome the deficiencies in the art.

It is a further object, feature, and/or advantage of any of the aspects and/or embodiments of the present disclosure to provide glasses that can be used with neuroimaging machines. For example, the glasses will be non-ferromagnetic.

It is still yet a further object, feature, and/or advantage of any of the aspects and/or embodiments of the present disclosure to provide glasses for neuroimaging machines that reduce fogging while wearing.

It is another object, feature, and/or advantage of any of the aspects and/or embodiments of the present disclosure to have glasses for use with a neuroimaging machine that allows a head covering portion of the machine to be positioned closely to the participant's head while mitigating contact and pressure on the glasses.

The glasses and systems disclosed herein can be used in a wide variety of applications. For example, while MEG and OPM machines are disclosed, it should be appreciated that any machine or device that includes a head covering where a participant may need to wear glasses in a comfortable manner could be used.

It is preferred that the glasses be safe, cost effective, and durable.

At least one embodiment disclosed herein comprises a distinct aesthetic appearance. Ornamental aspects included in such an embodiment can help capture a consumer's attention and/or identify a source of origin of a product being sold. Said ornamental aspects will not impede functionality of the glasses and/or glasses/machine combination.

According to some aspects of the present disclosure, a glasses frame for use with a neuroimaging machine, wherein the neuroimaging machine includes a head covering portion with a curved frontal opening, comprises two lens holders and a bridge connecting the two lens holders; each of the two lens holders comprising an outer rim and a cavity; wherein the two lens holders comprise an upper curvature forming an obtuse angle to conform with the curved front opening of the head covering portion of the neuroimaging machine; and wherein the frames comprise a non-ferromagnetic material.

According to at least some aspects of the present disclosure, the glasses frame further comprises at least one optical lens positioned in the cavity of one of the two lens holders.

According to at least some aspects of the present disclosure, the thickness between a top of the frames and a top of the cavity of the frame is equal or less than about 0.35 cm.

According to at least some aspects of the present disclosure, a width of the glasses frame is equal or less than about 12.6 cm.

According to at least some aspects of the present disclosure, a widest part of the frames is no higher than 1.4 cm from the top of the frames.

According to at least some aspects of the present disclosure, a width of the glasses frame at 0.5 cm below a top of the glasses frame is equal to or less than 9.5 cm.

According to at least some aspects of the present disclosure, a width of the glasses frame at 1 cm below a top of the glasses frame is equal to or less than 11 cm.

According to at least some aspects of the present disclosure, a width of a gap of the glasses frame between the two lens holders at about 1.75 cm below a top of the glasses frame is equal to or more than 1.4 cm.

According to at least some aspects of the present disclosure, a width of a gap of the glasses frame between the two lens holders at about 2.4 cm below a top of the glasses frame is equal to or more than 2.4 cm.

According to at least some aspects of the present disclosure, a height of the glasses frame is about 2.5 cm or more.

According to at least some aspects of the present disclosure, the glasses frame further comprises a retaining member comprising an adjustable strap connected to opposite sides of the glasses frame.

According to additional aspects of the disclosure, in combination, a glasses frame and neuroimaging device, comprises the neuroimaging device comprising a head covering portion with a curved frontal opening; and the glasses frame comprising two lens holders and a bridge connecting the two lens holders, each of the two lens holders comprising an outer rim and a cavity, wherein the two lens holders comprise an upper curvature forming an obtuse angle to conform with the curved front opening of the head covering portion of the neuroimaging machine, and wherein the frames comprise a non-ferromagnetic material.

According to at least some aspects of the present disclosure, the neuroimaging device comprises a magnetoencephalography (MEG) machine.

According to at least some aspects of the present disclosure, the neuroimaging device comprises an optically pumped magnetometer (OPM) machine.

According to at least some aspects of the present disclosure, a width of a gap of the glasses frame between the two lens holders at about 1.75 cm below a top of the glasses frame is equal to or more than 1.4 cm.

According to at least some aspects of the present disclosure, a width of a gap of the glasses frame between the two lens holders at about 2.4 cm below a top of the glasses frame is equal to or more than 2.4 cm.

According to at least some aspects of the present disclosure, the combination further comprises a retaining member comprising an adjustable strap connected to opposite sides of the glasses frame.

According to additional aspects of the disclosure, a pair of glasses comprises a frame comprising: two lens holders and a bridge connecting the two lens holders; each of the two lens holders comprising an outer rim and a cavity; wherein the two lens holders comprise an upper curvature forming an obtuse angle to conform with the curved front opening of the head covering portion of the neuroimaging machine; and wherein the frames comprise a non-ferromagnetic material; wherein a width of a gap of the glasses frame between the two lens holders at about 1.75 cm below a top of the glasses frame is equal to or more than 1.4 cm; and a retaining member comprising an adjustable strap connected to opposite sides of the glasses frame.

According to at least some aspects of the present disclosure, the pair of glasses further comprises at least one lens in one of the two lens holders.

According to at least some aspects of the present disclosure, the adjustable strap of the retaining member comprises a slidable locking member to adjust the size of the adjustable strap.

These and/or other objects, features, advantages, aspects, and/or embodiments will become apparent to those skilled in the art after reviewing the following brief and detailed descriptions of the drawings. The present disclosure encompasses (a) combinations of disclosed aspects and/or embodiments and/or (b) reasonable modifications not shown or described.

### BRIEF DESCRIPTION OF THE DRAWINGS

Several embodiments in which the present disclosure can be practiced are illustrated and described in detail, wherein like reference characters represent like components throughout the several views. The drawings are presented for exemplary purposes and may not be to scale unless otherwise indicated.

FIG. 1 is a figure showing an example of a MEG machine with a participant positioned with a helmet portion covering the head of the participant.

FIG. 2 is a depiction of prior art glasses used with a head covering such as used with a neuroimaging machine.

FIG. 3 is a depiction of another prior art glasses used with a head covering such as used with a neuroimaging machine.

FIG. 4 is a depiction of glasses according to aspects of the present disclosure used with a head covering such as used with a neuroimaging machine.

FIG. 5 is a perspective view of glasses for use with a neuroimaging machine according to aspects of the present disclosure.

FIG. 6 is a front elevation view of glasses for use with a neuroimaging machine according to aspects of the present disclosure.

FIG. 7 is a rear perspective view of glasses for use with a neuroimaging machine according to aspects of the present disclosure.

FIG. 8 is a top elevation view of glasses for use with a neuroimaging machine according to aspects of the present disclosure.

FIG. 9 is a side elevation view of glasses for use with a neuroimaging machine according to aspects of the present disclosure.

FIG. 10 is a depiction of glasses according to the present disclosure with retaining members.

FIG. 11 is a side elevation view of FIG. 10.

FIG. 12 is a view of an example helmet with sensors used with an OPM.

An artisan of ordinary skill in the art need not view, within isolated figure(s), the near infinite distinct combinations of features described in the following detailed description to facilitate an understanding of the present disclosure.

### DETAILED DESCRIPTION

Unless defined otherwise, all technical and scientific terms used above have the same meaning as commonly understood by one of ordinary skill in the art to which embodiments of the present disclosure pertain.

The terms “a,” “an,” and “the” include both singular and plural referents.

The term “or” is synonymous with “and/or” and means any one member or combination of members of a particular list.

As used herein, the term “exemplary” refers to an example, an instance, or an illustration, and does not indicate a most preferred embodiment unless otherwise stated.

The term “about” as used herein refers to slight variations in numerical quantities with respect to any quantifiable variable. Inadvertent error can occur, for example, through use of typical measuring techniques or equipment or from differences in the manufacture, source, or purity of components.

The term “substantially” refers to a great or significant extent. “Substantially” can thus refer to a plurality, majority, and/or a supermajority of said quantifiable variables, given proper context.

The term “generally” encompasses both “about” and “substantially.”

The term “configured” describes structure capable of performing a task or adopting a particular configuration. The term “configured” can be used interchangeably with other similar phrases, such as constructed, arranged, adapted, manufactured, and the like.

Terms characterizing sequential order, a position, and/or an orientation are not limiting and are only referenced according to the views presented.

The “scope” of the present disclosure is defined by the appended claims, along with the full scope of equivalents to which such claims are entitled. The scope of the disclosure is further qualified as including any possible modification to any of the aspects and/or embodiments disclosed herein which would result in other embodiments, combinations, subcombinations, or the like that would be obvious to those skilled in the art.

The present disclosure is not to be limited to that described herein. Mechanical, electrical, chemical, procedural, and/or other changes can be made without departing from the spirit and scope of the present disclosure. No features shown or described are essential to permit basic operation of the present disclosure unless otherwise indicated.

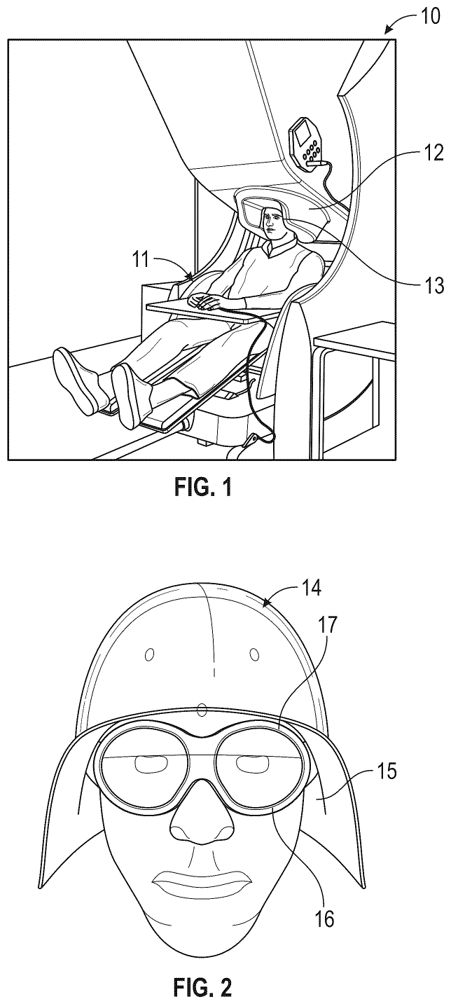

FIG. 1 is a depiction of a magnetoencephalography (MEG) machine, which is a neuroimaging machine 10. A participant 11 is engaged with the machine to allow the machine to acquire neural data, such as through a helmet or head covering 12. A MEG, such as the one shown in FIG. 1, is a functional neuroimaging technique for mapping brain activity by recording magnetic fields produced by electrical currents occurring naturally in the brain, using very sensitive magnetic sensors. Each of these sensors are coupled to a single SQUID (superconducting quantum interference devices), which transforms the magnetic field into a voltage measurement that can be recorded by conventional electronics. Applications of MEG include basic research into perceptual and cognitive brain processes, localizing regions affected by pathology before surgical removal, determining the function of various parts of the brain, and neurofeedback. This can be applied in a clinical setting to find locations of abnormalities as well as in an experimental setting to simply measure brain activity.

While a MEG is shown in FIG. 1, it should be noted that other neuroimaging machines 10, such as optically-pumped magnetometry-magnetoencephalogram (OPM-MEG) systems and the like are considered a part of the disclosure, and the term “neuroimaging system” or “machine” will be used to cover any type of neuroimaging system that is known and used to measure brain activity.

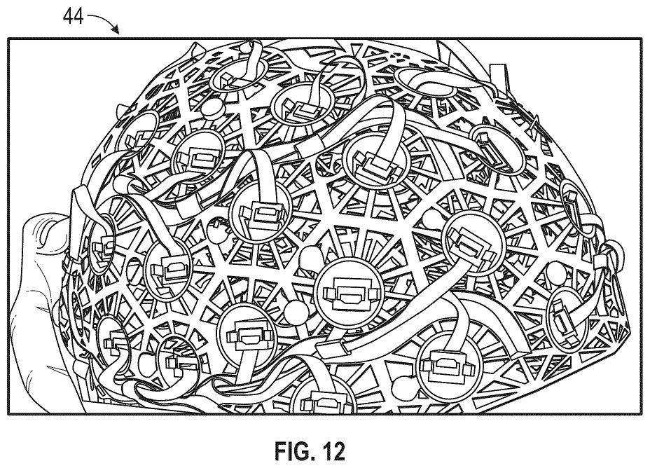

As is shown with the MEG 10 of FIG. 1, as well as the OPM helmet 44 of FIG. 12, the devices include portions that go on or over a portion of a participant's head. It is important for the helmets or head coverings (e.g., head covering 12 or helmet 44) to be positioned as closely to the head of the participant as possible to be able to best utilize the machine to acquire information. Generally, the machines are used with a participant while the participant performs activities so that the brain activity can be monitored and measured for research and other purposes. Some of these activities involve the participant needing to see and then react or act. This becomes especially key when the participant requires the use of glasses or other optics to aid in seeing.

For MEGs, any glasses that are used with the machine must have no ferromagnetic metal. This means that regular glasses with hinges and screws cannot be used. Additionally, the normal shape of most glasses are too large and bulky to be able to fit a participant's head comfortably while in the MEG. Most non-ferromagnetic glasses are made to be able to work with MRIs which do not have a shape restriction. Most MEGs have a smaller window that glasses can fit in which requires the glasses to have a sharp curve down on the top of the frames. For adults or children with smaller heads, their eyes are level with the MEG's rim, so the glasses need to sit low on the face. If the glasses obstruct the ability for the person's head to reach the top of the helmet of the scanner, the scanner is unable to get the best data. A further issue with regular glasses is the legs of the glasses. Often participants being scanned in the MEG have head position indicator coils (HPI coils) taped behind their ears so glasses that have legs that bend around the ear may touch or move the HPI coils causing noise in the signal of the MEG.

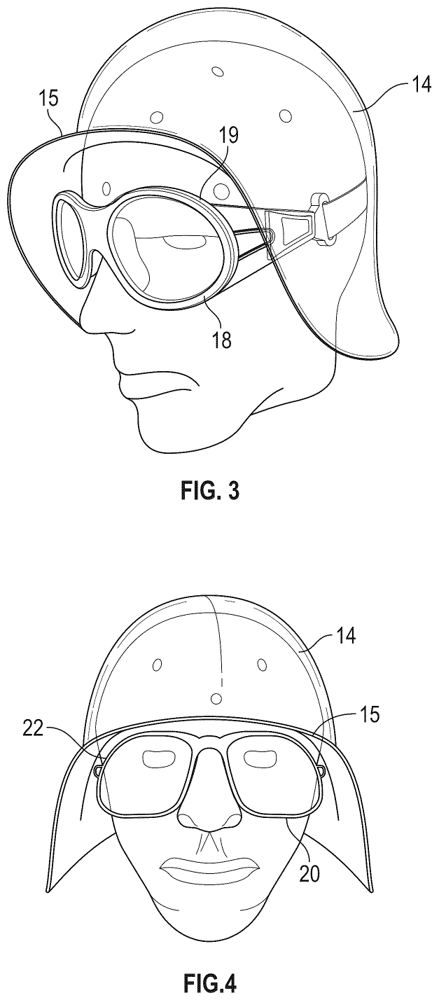

Some attempts to address the issue of wearing glasses with neuroimaging machines, such as shown in FIG. 1, are shown in FIGS. 2 and 3. For example, FIGS. 2 and 3 show prior art glasses made of non-ferromagnetic materials. FIG. 2 shows a helmet 14 (e.g., one that could be used with a neuroimaging device) that includes a front curved opening 15. The glasses 16 shown include a frame 17. However, the shape of the frame 17 of the glasses 16 interact with the front opening 15 of the helmet to prevent the helmet from being as close as needed to the participant's head. In addition, glasses such as these tend to fog up during use, and therefore, would not be ideal for use.

Likewise, FIG. 3 shows prior art glasses 18 with the same helmet 14 as FIG. 2. Again, the shape of the frame 19 of the glasses 18 prevent the helmet 14 from being positioned at or near the head of the participant.

Therefore, as shown in FIGS. 4-11, a pair of glasses 20 are provided that overcome many of the issues of the prior art and also provide numerous advantages for use with neuroimaging machines, including those with helmets or head coverings. As will be understood, to address the issues of the prior art, the glasses 20 of the present disclosure have sharp curves along the top edges of the frames, there is a maximum width the frames can be, and the outer rim of the frame, specifically on the top, is also thin to minimize the area that the person cannot see out of while still allowing the frame to not break and support the lens and itself. The nose piece is also wide enough to allow the glasses to sit lower on the face.

As shown in FIG. 4, the glasses 20 fit comfortably on a participant to allow the helmet 14 to be fully positioned near the head so the sensors can best acquire the needed information. The frame 22 of the glasses 20 are curved based on the opening 15 of the helmet 14 to mitigate contact therewith. This will allow the helmet to fully fit on the head and also mitigate fogging of the glasses.

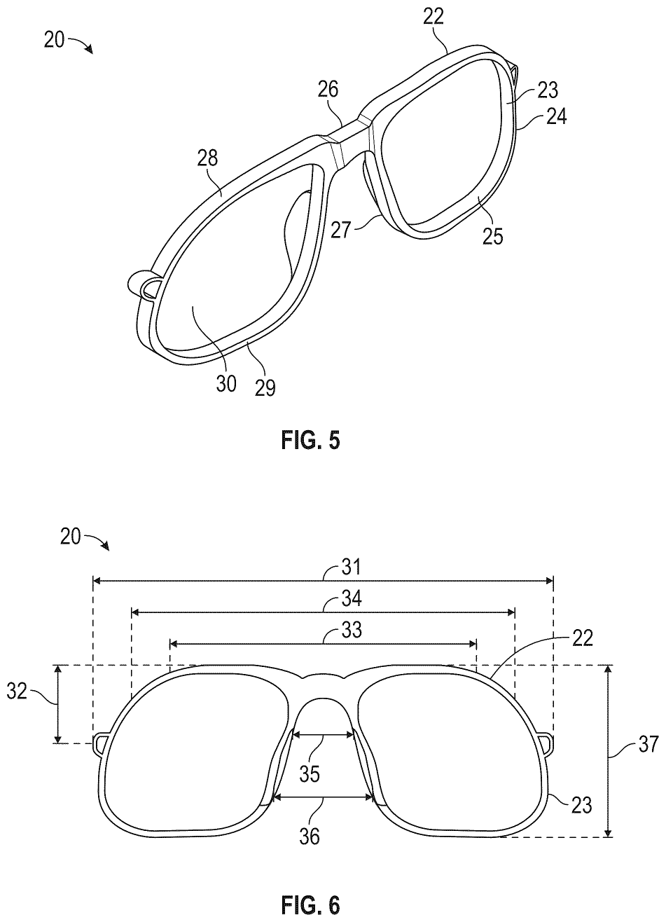

Moving to FIG. 5, a perspective view of the glasses 20 of the present disclosure are shown according to at least some aspects and/or embodiments of the disclosure. The glasses 20 include a frame 22 including at least one optical lens 30 to allow a participant to see. For example, each or both of lens holders 23 of the frame can be fit with the same or different prescriptions of optical lens for users to be able to better see while wearing the glasses 20 with the neuroimaging machine 10.

The frame 22 may be 3D printed (or otherwise created using additive manufacturing), sculpted, carved, cut, molded, or formed out of any solid body or body that will harden into a solid body that is not magnetic. Each frame 22 comprises of two lens holders 23 and one bridge 26 that connects the two lens holders 23. The bridge 26 is the connecting piece that holds the two lens holders 23 together. The bridge 26 can also allow a resting place for the nose for those who have smaller noses. Note there is also nose guards 27 that can contact larger noses. Each lens holder 23 has an outer rim 24 and a cavity 25 to hold the lens 30 inside the rim 24.

Further, note that the following orientation references will be given with respect to the glasses in order to better understand some of the dimensional improvements of the glasses. The frame 22 has a top 28 and a bottom 29. Note that these are not to be limiting terms and are provided for disclosure purposes to understand the scope of the disclosure.

Moving to FIG. 6, note that there are multiple dimensional arrows provided. As will be understood, the present disclosure includes dimensional aspects of the glasses frame 22 that will provide improvements and/or advantages over the prior art, and therefore, the glasses frame will be disclosed with respect to said dimensions.

For example, a preferred maximum thickness of the top 28 of the frame's cavity (see, e.g., FIG. 8) for the lenses should be no more than 0.35 cm. The thickness should be thin to mitigate restricting a participant's view if the person requires to see at of the very top portion of the glasses. Participants with shorter foreheads or smaller heads would need to see at the very top portion of the glasses because when their head is inside the helmet 14 their eye line is almost obstructed by the rim of the MEG.

The width of the glasses (shown by arrow 31 in FIG. 6) is preferred to be no more than 12.6 cm. Many helmet shapes are conforming to a person's head to get the best signals from the sensors. This means there is often little space to extend the glasses frame 22 much wider than the eyes themselves. Any wider than 12.6 cm would limit the amount of helmet shapes the frames can fit in.

The widest part of the frame 22 is preferred to be no higher than 1.4 cm from the top 28 of the frame (see, e.g., arrow 32 in FIG. 6). It is common for helmet shapes to be thinner at the top of the cutout for the eyes and then widen as it gets closer to the cheeks. Having the widest part higher than 1.4 cm on the glasses frame 22 would reduce the amount of helmet shapes the glasses can fit in.

The width of the frame 22 0.5 cm from the top 28 is preferred to be 9.5 cm or less (see, e.g., arrow 33 in FIG. 6). The width 1 cm from the top 28 is preferred to be no more than 11 cm (see, e.g., arrow 34 of FIG. 6).

The width of the gap (see, e.g., arrow 35 in FIG. 6) between the lens holders 23 (where the nose sits), 1.75 cm from the top 28, is preferred to be no less than 1.4 cm. It is beneficial to have the place for the nose to be larger than normal glasses. If the space for the nose is too small the glasses sit on top of the nose making the frames 22 sit higher than they should. If the glasses are too high on the face, they are unlikely to fit in the helmet.

In addition, the width of the gap (see, e.g., arrow 36 in FIG. 6) between the lens holders 23 (where the nose sits), 3 cm (if the glasses are that tall) from the top 28, is preferred to be no less than 2.4 cm. The gap between the lens holders (where the nose sits) is preferred to get wider the farther from the top of the frame 28. The place where the nose sits should get wider as the nose gets wider so that farther from the top of the glasses the wider the gap for the nose should be.

The overall height of the glasses is preferred to be at least 2.5 cm (see, e.g., the arrow 37 in FIG. 6). The overall height of the lenses 30 should be large enough to not be distracting when looking through them. Because of that, it is recommended that having the lenses no shorter than 3 cm but at least 2.5 cm so the frame 22 would still be able to properly sit on/hug the nose.

It is optional but recommended to have nose pieces 27 that give the frames 22 more surface area rest on the nose.

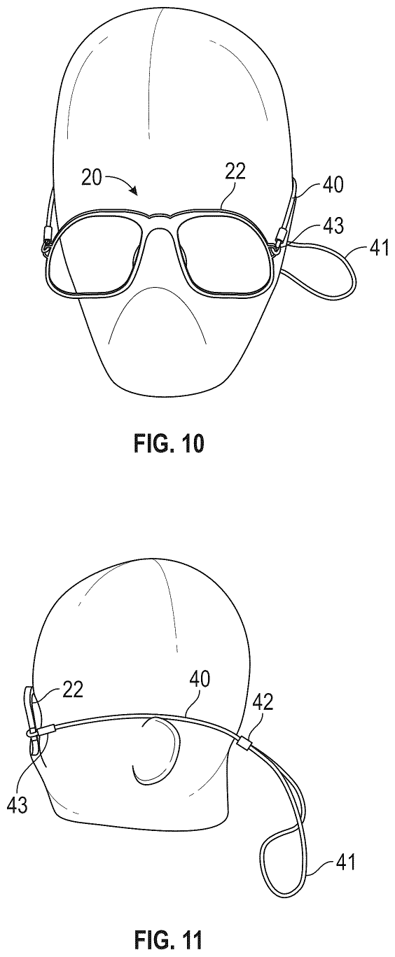

The frames 22 can balance on the nose with the help of the larger opening that the frames sit on the nose, but it is recommended that a flexible thin material is attached to both ends and can be tied or sinched behind the head to adjust the slack in the material to hold them on the head. Such a retaining member 40 is shown in FIGS. 10 and 11. It is preferred to not use elastic because it cannot be easily adjusted once the person is in the helmet which means it loses some functionality of being able to sit low on the face. Therefore, as shown in FIGS. 10 and 11, a strap 41 with a slidable adjuster 42 allows the strap 41 to be size-adjustable and easily adjusted, even when the participant's head is positioned in the covering/helmet. In addition, note that the straps 41 can be attached to the frame 22 at attachment members 43, which are protrusions with apertures to allow connecting of the strap to the frame. However, additional ways of attaching a strap or other retaining member to the frame are to be considered part of the disclosure.

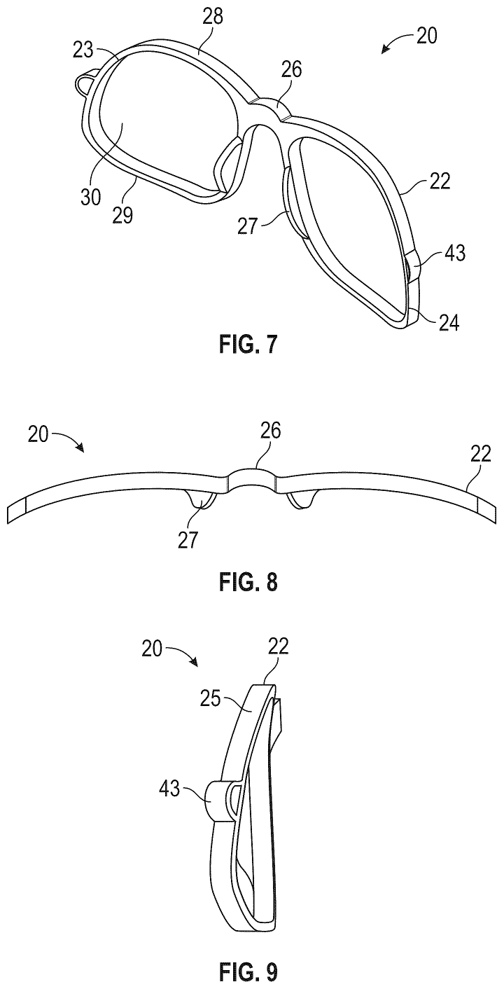

FIGS. 7-9 show additional aspects of the glasses 20 of the disclosure. For example, it is optional to curve the frames 22 to the face. A slight curve allows the outer rim, or edge, of the frame 22 to disappear into the peripheral vision, but too much curve towards the face would cause the frames 22 to hold tight to the face which would cause the lenses to fog because of the lack of airflow.

The edge of the frame 22 tangential to the edge of the lens should not stick out from the lenses 30 more than 0.5 cm for more than 50% of the lens holder. This is to allow air flow around the lenses, so the lenses do not fog up.

At least one optical lens 30 is placed in one of the cavities 25 of the frames 22, but the glasses 20 may have two lenses 30 placed with one in each of the cavities in the frames. The lens(es) 30 may be permanent or removable within the frames 22. Permanently fitting the lens(es) 30 in the frames 22 means there is less wear and tear to the frames 22 and the pair of glasses is likely to last longer, but removable lenses allow for the purchase or manufacturing and storing of only one frame 22. For example, having removable lenses would allow for fewer frames and more adjustability for participants who need visual aid during the interaction with the neuroimaging device.

The lens(es) 30 may be friction fit, chemically bonded, physically bonded, thermally fit, or may be balanced in place with at least one surface. If a removable lens is preferred it is recommended, but not necessary, to have a beveled divot around the entire inner ring of the lens holders. The bevel would allow the lens(es) 30 to be balanced in place with two surfaces instead of only one.

Therefore, as can be appreciated, the glasses 20 shown and/or described provide numerous benefits and/or advantages. The wide nose area is at least one novel portion of the glasses 20. Without the wide nose portion, the glasses 20 are less able to sit lower on the face of a participant. The combination of the restricted width of the glasses, the thinness of the top of the lens holders, wide nose area, and allowing airflow around the frames and face are what make these glasses different than all the other brands.

These frames 22 of the glasses 20 can sit on the face lower to allow the head to reach the top of the helmet without interference from the frames. The lenses 30 have a low tendency to fog up due to the shape of the frames 22 of the glasses 20 that allows airflow when worn by a participant. When in the covering/helmet, the participant can adjust where the glasses 20 sit on their face via the strap 41 with a slidable adjuster 42 to where they find it more comfortable. There are no legs that wrap behind the ears that would interfere with where the head position interface coils (HPI coils) could be located.

While MEG and OPM machines have been identified, it should be noted that these are not the only machines the glasses could be used with. One use for these glasses is with neuroimaging and neurostimulation equipment. Examples include but are not limited to: MRI, MEG, EEG, OPM, TMS, tDCS. Secondary uses are uses where optical glasses are required with a helmet or hat. Tertiary use includes anyone that is required to be magnetically silent to work.

Therefore, glasses for use with helmets or other head coverings have been shown and/or described. It should be appreciated that variations and/or changes to any of the components or embodiments that are obvious to those skilled in the art are to be considered a part of the present disclosure. In addition, any of the aspects of any of the embodiments disclosed could be combined in ways not explicitly shown and/or described to provide yet additional embodiments that are part of the disclosure. The disclosure is not to be limited to the embodiments disclosed herein.

1. A glasses frame for use with a neuroimaging machine, wherein the neuroimaging machine includes a head covering portion with a curved frontal opening, the glasses frame, comprising:
two lens holders and a bridge connecting the two lens holders;
each of the two lens holders comprising an outer rim and a cavity;
wherein the two lens holders comprise an upper curvature forming an obtuse angle to conform with the curved front opening of the head covering portion of the neuroimaging machine; and
wherein the frames comprise a non-ferromagnetic material.

2. The glasses frame of claim 1, further comprising at least one optical lens positioned in the cavity of one of the two lens holders.

3. The glasses frame of claim 1, wherein the thickness of the frame from a top of the frame to a top of the cavity of the frame is equal to or less than about 0.35 cm.

4. The glasses frame of claim 1, wherein a width of the glasses frame is equal or less than about 12.6 cm.

5. The glasses frame of claim 1, wherein a widest part of the frames is no higher than 1.4 cm from the top of the frames.

6. The glasses frame of claim 1, wherein a width of the glasses frame at 0.5 cm below a top of the glasses frame is equal to or less than 9.5 cm.

7. The glasses frame of claim 1, wherein a width of the glasses frame at 1 cm below a top of the glasses frame is equal to or less than 11 cm.

8. The glasses frame of claim 1, wherein a width of a gap of the glasses frame between the two lens holders at about 1.75 cm below a top of the glasses frame is equal to or more than 1.4 cm.

9. The glasses frame of claim 8, wherein a width of a gap of the glasses frame between the two lens holders at about 2.4 cm below a top of the glasses frame is equal to or more than 2.4 cm.

10. The glasses frame of claim 1, wherein a height of the glasses frame is about 2.5 cm or more.

11. The glasses frame of claim 1, further comprising a retaining member comprising an adjustable strap connected to opposite sides of the glasses frame.

12. In combination, a glasses frame and neuroimaging device, comprising:
the neuroimaging device comprising a head covering portion with a curved frontal opening; and
the glasses frame comprising two lens holders and a bridge connecting the two lens holders, each of the two lens holders comprising an outer rim and a cavity, wherein the two lens holders comprise an upper curvature forming an obtuse angle to conform with the curved front opening of the head covering portion of the neuroimaging machine, and wherein the frames comprise a non-ferromagnetic material.

13. The combination of claim 12, wherein the neuroimaging device comprises a magnetoencephalography (MEG) machine.

14. The combination of claim 12, wherein the neuroimaging device comprises an optically pumped magnetometer (OPM) machine.

15. The combination of claim 12, wherein a width of a gap of the glasses frame between the two lens holders at about 1.75 cm below a top of the glasses frame is equal to or more than 1.4 cm.

16. The combination of claim 15, wherein a width of a gap of the glasses frame between the two lens holders at about 2.4 cm below a top of the glasses frame is equal to or more than 2.4 cm.

17. The combination of claim 12, further comprising a retaining member comprising an adjustable strap connected to opposite sides of the glasses frame.

18. A pair of glasses, comprising:
a frame comprising:
two lens holders and a bridge connecting the two lens holders;
each of the two lens holders comprising an outer rim and a cavity;
wherein the two lens holders comprise an upper curvature forming an obtuse angle to conform with the curved front opening of the head covering portion of the neuroimaging machine; and
wherein the frames comprise a non-ferromagnetic material;
wherein a width of a gap of the glasses frame between the two lens holders at about 1.75 cm below a top of the glasses frame is equal to or more than 1.4 cm; and
a retaining member comprising an adjustable strap connected to opposite sides of the glasses frame.

19. The pair of glasses of claim 18, further comprising at least one lens in one of the two lens holders.

20. The pair of glasses of claim 18, wherein the adjustable strap of the retaining member comprises a slidable locking member to adjust the size of the adjustable strap.
